(19) United States
(12) Patent Application Publication
(10) Pub. No.: US 2009/0232760 A1
(43) Pub. Date: Sep. 17, 2009

(54) Horse Bedding Product and Method

(76) Inventor: Ross McArthur, Encinitas, CA (US)

Correspondence Address: GEORGE S. LEVY, 3980 DEL MAR MEADOWS, SAN DIEGO, CA, 92130, US

(21) Appl. No.: 12/048,980

(22) Filed: Mar. 14, 2008

(51) Int. Cl. A61L 9/01; A01K 29/00

(52) U.S. Cl. 424/76.1; 119/171

(57) ABSTRACT

A layered bedding configuration installed in a stall for an animal for reducing odors. It comprises:
a) a first film of enzyme solution on the floor walls;
b) a first layer of enzymatic-bacterial composition on top of the first film of enzyme solution;
c) a layer of rice hulls on top of the first layer of enzyme powder, the layer of rice hulls being gradually crushed as the animal moves in the stall;
d) a second layer of enzyme powder;
e) a second film of enzyme solution on top of the second layer of enzyme powder,
   wherein the enzyme powder comprises hydrolyzing enzymes, bacteria and bacterial growth medium and wherein the enzyme solution and the enzyme powder act on fecal and urinary materials to reduce odor, and the layer of rice hulls increases its absorbency as a function of the degree that it is crushed by the animal.

### FIELD OF THE INVENTION

The present invention relates to bedding materials used in horse stalls as well as the method used for the application of these materials. More specifically it relates to bedding materials comprising rice hulls as well as enzymes that reduce odor.

### INCORPORATION BY REFERENCE

European patent EP0878202 by Bonassi, and European patent EP0808564 by Pedemonte are hereby incorporated by reference.

### BACKGROUND

Bedding for horses traditionally employs materials which are industrial by-products. These materials have in recent years been diverted for other uses by the industries that produce them. For example, shavings and sawdust, by-products of the lumber industry have been a major source of horse bedding for many years. In addition, the lumber industry has increased its demand for these materials to fuel their boiler system. Furthermore, these products are being used as a raw material for the production of methane as a substitute for gasoline. Now because of the greater need for these products by the building industry for the manufacture of pressed wood products, these materials are less available to be used as horse bedding.

Examples of materials used for bedding include the traditional straw, shavings and hay. Also included in this group of materials are paper, cardboard, peanut hulls, rice hulls, tobacco stems, corn fodder, bark, chopped corn-cobs, and tanbark.

According to current prior art, criteria other than cost, availability and transportation, that should govern the selection of bedding material includes absorbency, dustiness, padding ability, allergenicity, adherence to shoes or hoofs, toxicity when eaten, handling and storage requirements, chemical stability, and flammability of the materials (Reference: AS39, one of a series of the Animal Science Department, Florida Cooperative Extension Service, Institute of Food and Agricultural Sciences, University of Florida. Original publication date November 1993. Reviewed June 2003. The author is E. L. Johnson, Assistant Professor, Department of Animal Science, Cooperative Extension Service, Institute of Food and Agricultural Sciences, University of Florida, Gainesville, 32611.

Thus, according to prior art, absorbent materials are desirable for bedding. Yet this characteristic is double edged. Absorption and retention of moisture laden with organic products encourages the growth of bacteria.

Bedding materials vary greatly in their absorbency, with rice hulls and hardwood products being least absorbent. Pine wood shavings and barley and wheat straw are moderately absorbent, while pine wood sawdust and chips are more absorbent. Long oat straw is the most absorbent type of straw; shredded newspaper is more absorbent than any straw or wood product; and peat moss is the most absorbent type of bedding. (Reference: Thoroughbred Times Jun. 24, 2000).

Currently, the majority of conventional horse bedding consists of a layer of wood shavings spread on the floor of the stall. Waste material is removed with a fine tine pitchfork and the bedding material is changed periodically. Yet this kind of bedding material has over the years become more problematic to procure and more expensive because of increasing demand for wood by-products. A typical expense today for supplying and maintaining bedding for a horse approximately ranges from $1500 to $3000 per year.

The maintenance of such bedding typically requires the removal of solid waste material with a pitchfork and the rinsing of urine with a water hose. Because of the propensity of wood shavings to absorb moisture, bacterial growth is enhanced resulting in unpleasant odors and unsanitary conditions in horse stalls.

European patent EP0878202 by Bonassi, assigned to Eurovix, an Italian company, describes a product for sanitizing and deodorizing curbside bins for collecting waste in general. It comprises a mix of enzyme powders and bacteria powders with mineral salts. It also comprises a culture medium for the bacterial strains of the bacteria powders. This product is manufactured by Eurovix an Italian company.

European patent EP0808564 by Pedemonte describes bedding for stalled animals, and in particular for horses, which includes a homogeneous mixture of wood shavings and an enzymatic-bacterial composition including at least one class of enzymes or their precursors and at least one bacterial species. This patent is assigned to AGRICOMBI an Italian company. This product contains α-amylase, β-amylase, pentosanase, lipase, glucoamylase, cellulase, hemicellulase, lactase, protease, lipase, phosphorylase, pectinase, β-lactamase, and pullulanase; or their precursors. It is distributed by the Italian company Eurovix.

There is a need for a less expensive, more available, “non-allergenic” and more hygienic material for horse bedding. In addition there is a need for a method for setting up and maintaining horse bedding comprised of such material.

None of the prior art offers the economy and hygiene of the present invention. Further features, aspects, and advantages of the present invention over the prior art will be more fully understood when considered with respect to the following detailed description and claims.

### BRIEF DESCRIPTION OF THE DRAWINGS

FIG. 1 represents a stall with bedding and a horse on it. A board is shown on its edge to retain the rice hulls inside the stall.

FIG. 2 is a cross section of the bedding showing from the floor up, a bottom layer of enzyme powder, the rice hulls, and a top layer of enzyme powder.

FIG. 3 illustrates the growth and removal of a wet spot in the bedding shown in cross section.

### SUMMARY OF THE INVENTION

1 This invention is a layered bedding configuration installed in a stall to be used as a shelter for an animal such as a horse. This bedding configuration is designed to reducing odors. This invention also covers the method of construction and the method of maintenance of this bedding configuration. It comprises:
   a) a film of enzyme solution sprayed on the floor and on the walls of the stall;
   b) a layer of enzymatic-bacterial composition called enzyme powder spread on top of the first film of enzyme solution;
   c) a layer of rice hulls spread on top of the first layer of enzyme powder. As this layer of rice hulls is being gradually crushed by as the animal moves in the stall, the rice hulls increase their absorbency thereby making the bedding more absorbent the more it is used.
   d) a second layer of enzyme powder is applied on top of the rice hulls;
   e) a second film of enzyme solution is applied on top of the second layer of enzyme powder;
      The enzyme powder comprises hydrolyzing enzymes, bacteria and bacterial growth medium. In addition the enzyme solution and the enzyme powder act on fecal and urinary materials produced by the animal to reduce odor, and the layer of rice hulls increases its absorbency as a function of the degree that it is crushed by the animal, and therefore as a function of the time the animal occupies the stall.

### DETAILED DESCRIPTION

This invention is a layered bedding configuration for horse stalls. This bedding consists of a non-homogeneous composition of rice hulls and enzymatic/bacterial powder and liquid as well as a method of maintaining this bedding material, which maximizes it useful life and minimizes expenses. The enzyme powder and a liquid enzyme liquid solution are both manufactured in the United States. The powder is manufactured by Specialty Enzymes in Chino, Calif. and is sold under the name “EnviroSEB™”, and the liquid enzyme solution is manufactured by Enzyme Solutions in Fort Wayne Ind. and is sold under the name of “Biomagic™”.

Rice Hulls: rice hulls have the advantage of being a natural product and a by-product of rice cultivation. They do not absorb moisture unless they are crushed and therefore are less supportive of bacterial growth especially if the rice hulls are used in conjunction with the enzyme powder and the enzyme liquid. Another advantage of rice hulls is that because their absorbency increases as a function of the degree that they are crushed by the horse moving around in the stall, they are used in proportion to the time that the horse spends on the bedding.

Enzyme Powder: the enzyme powder comprises a combination of hydrolyzing enzymes, selected bacteria and minerals. One version of this powder is utilized in the European patent EP0808564 by Pedemonte which has been incorporated by reference. A second version of this powder is sold by Specialty Enzymes and Eurovix and comprises the following ingredients:
a) Enzymes: Alpha Amylase, Beta Amylase, Pentosanase, Gluco-Amylase, Cellulase, Lactase, Pancrease, Protease, Phosphorilase, Pullulanase, Hemicellulase, Pectynase, Beta-glucanase;
b) Minerals: Mineral salts of Calcium and Magnesium, Mordenite mineral salts, Dolomia mineral salts;
c) Selected bacteria: Micrococcus, Bacillus;
d) Growth medium: maltodextrin, AGAR broth medium, Seaweed Lithothamnium Calcareum, DNA and RNA nucleic acids, Active principles (iodine) of Laminariae Stipites (Kelp), and Fucus Vesiculosus (Bladderwrack);

Enzyme Solution: The Technical Data Sheet published by Eurovix for the liquid enzyme solution includes the following ingredients:
a) Enzymes: Protease, Amylase, Cellulase, Lipase, Hemicellulase, Beta Glucanase;
b) Selected microorganisms;
c) Physiologic solution 5%;
d) Glucose solution 5%;
e) Pigmented vegetable extract;
f) Natural Pine oil;

The company Enzyme Solutions Inc. is another supplier of enzyme solution. The product “BioMagic™ they supply comprises protease, lipase, carbohydrase, surfactants, and odor eliminating ingredients.

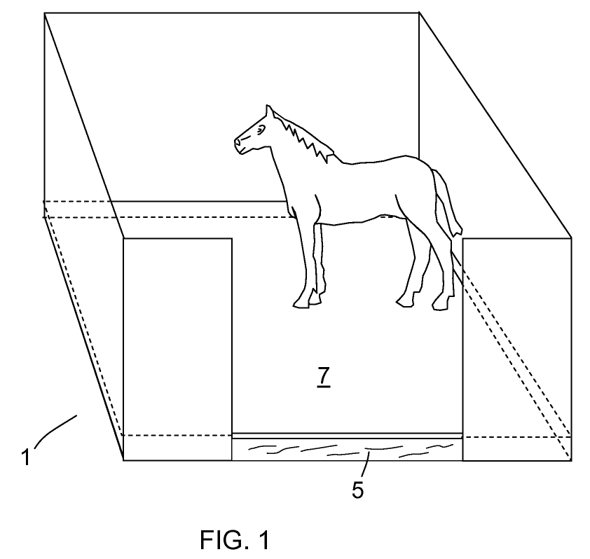

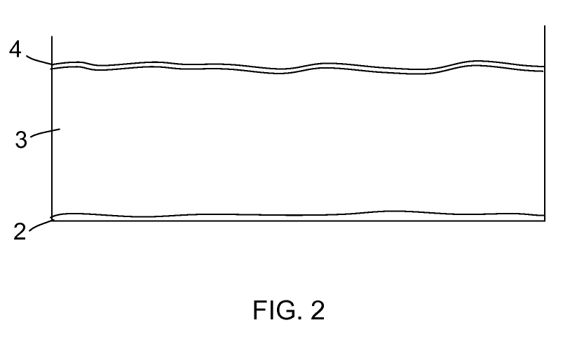

Bedding Constitution: FIGS. 1 and 2 illustrate the constitution of the bedding in a horse stall 1. For the purpose of quantifying the enzyme material needed, a stall 1 is defined as measuring approximately 12 feet by 12 feet. A larger stall will require proportionally larger amount of enzyme powder and enzyme liquid. The bedding 7 is comprised of:
a) 8 to 10 ounces of liquid enzymes sprayed on the floor and applied to the walls to a height of about 4 feet. The liquid is not shown in the Figures as it only wets the walls and the floor;
b) 7 to 10 ounces of powder enzyme 2 spread onto the floor;
c) Approximately 6 inches of rice hulls 3 evenly covering the floor of the stall 1 on top of the powdered enzyme layer 2. An interesting property of rice hulls in comparison with the conventionally used pine wood sawdust and chips is that they increase their absorbency when they are crushed. Since the animals crush the hulls as they walk around the stall, a bedding comprising rice hulls absorbs in proportion to the time of occupancy of the stall by the animal;
d) 8 to 10 ounces of powder spread as a layer 4 to the top of the rice hulls;
e) 8 to 10 ounces of liquid enzymes applied to the top of the bedding. The liquid is not shown in the Figures as it seeps through the bedding.
   It is clear to someone having skill in the arts that the liquid enzyme solution sprayed on the floor and on the top of the bedding partially or fully diffuses into the layers of enzyme powder and of rice hulls. Furthermore, the bottom and top layers of enzyme powder intermingle with the rice hulls because of the movements of the animal and because of the manipulation of the bedding by keepers when they remove fecal matter by rake or pitchfork.

Bedding Set Up Method: the initial setting up of the bedding comprises the following steps:
a) Initial clean up which consists of removing any old bedding material. Usually that bedding is a wood product such as sawdust or wood shavings.
b) Spraying the floor and the wall to a height of approximately 4 feet with the liquid solution of enzymes. This can be done for example with a conventional pressure sprayer.
c) Spreading to the floor a layer 2 consisting approximately of 5 ounces of enzyme powder for every 100 square feet. For example if the stall 1 is 12 feet by 12 feet, then about 7 ounces should be applied. A larger stall measuring for example 14 feet by 14 feet should receive about 10 ounces. If the stall 1 houses a gelding, the powder should be applied more abundantly in the center area of the stall. If the stall 1 houses a mare, the powder should be applied near the periphery of the stall 1.
d) Spreading rice hulls into the stall. There should be enough bedding 3 to cover the floor to a depth of approximately 6 inches. Transporting the rice hulls into the stall 1 can be done by means of a wheel-barrow. It can also be accomplished by means of drag mats. The drag mat method requires loading the hulls on top of a drag mat outside the stall, dragging the mat into the stall by pulling on the drag mat's handle, and lifting/rolling the mat to dump the hulls into the stall. Because of the low repose angle of the rice hulls (how steep they can form a hill) drag mats are more efficient at carrying rice hulls than wheel barrows. The angle of repose is also referred to as angle of friction and is a property of granular materials. It is the maximum angle of a stable slope determined by friction, cohesion and the shapes of the particles. Material with a low angle of repose forms flatter piles than material with a high angle of repose.
e) Placing a board 5 about 6 inches wide on its edge on the floor across the entrance of the stall 1 to prevent the hulls from spilling outside.
f) Evening out the hulls by means of a pitchfork or a rake.
g) Applying the liquid enzyme solution, for example by means of a sprayer.
h) Applying enzyme powder 4 to the top of the bedding 3.

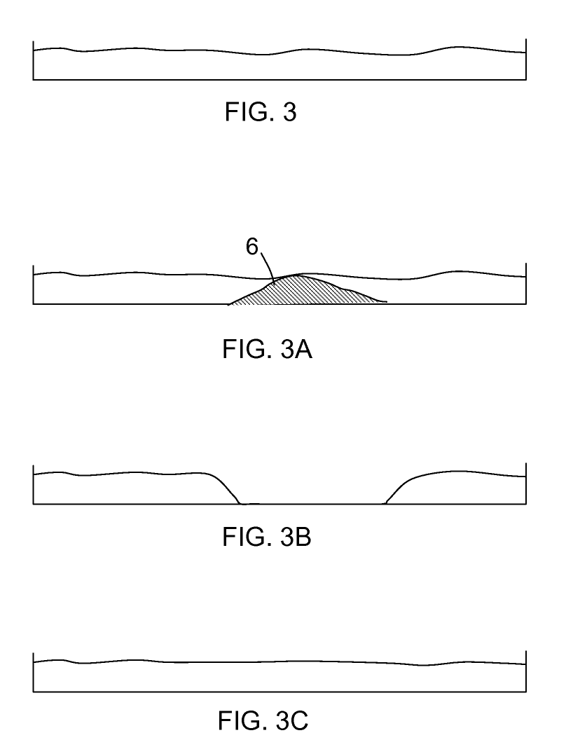

Maintenance method: After the initial installation is complete the bedding needs to be maintained to increase its useful life and to reduce odors. The following maintenance method assumes full occupancy of the stall by a horse. If occupancy is less frequent then the maintenance procedure may be done less often. FIG. 3 through FIG. 3C illustrate the general steps that are taken during maintenance. FIG. 3 shows the stall in its clean state before being occupied by a horse. FIG. 3A shows a wet spot 6 after a horse has urinated. FIG. 3B illustrates the state of the stall 1 after removal of the wet spot and FIG. 3C shows the stall 1 after the reconstitution of the bedding. The maintenance steps are:
a) Removing fecal material in the stall;
b) Adding rice hulls to compensate for the compaction of the rice hulls be the animal;
c) Spreading enzymes on top of the bedding configuration to restore the top layer of enzyme powder;
d) Spraying the walls and the top of bedding with the enzyme solution;
e) Removing wet spot which consist of bedding soaked with urine;
f) Add rice hulls to compensate for bedding material removed during clean up and for the compaction of the bedding by the animal;

The schedule below has been found to be economical and most effective in the maintenance of the bedding. It consists of the following steps:
a) Twice a day removing the fecal matter produced by the animal with a fine tine pitchfork by running a pitchfork through the bedding. This action also aerates the bedding and contributes to drying of the bedding and to reducing odors;
b) Every week adding about 12 cubic feet of rice hulls to compensate for the loss of bedding material during cleanup and for the compaction of the material by the horse.
c) Every week adding about 8 to 10 ounces of enzyme powder;
d) Once a month spraying 10 to 20 ounces, preferably about 15 ounces of liquid onto the bedding and over the walls to a height of about 4 feet;
e) Removing wet spots as required. This activity requires the following steps:
   i. Determining that a wet spot needs to be removed when it has grown to such a size that it is barely visible below the top surface of the bedding;
   ii. Exposing the wet spot by brushing away the dry surrounding material;
   iii. Removing only the wet material, making a hole into bedding and bedding and exposing the floor of the stall;
   iv. Spreading about 8 ounces of powder to the exposed floor;
   v. Raking into the hole and leveling the remaining bedding material.
   vi. Spreading about 12 cubic feet of hulls to compensate for the removal of the wet material and for the loss of volume caused by the compaction of the hulls by the horse;
   vii. spreading said enzyme powder on top of said layered bedding configuration to restore said second layer of enzyme powder.

The quantities of enzyme powder, enzyme solution and rice hulls have been assumed in this document to be suitable for a standard stall having nominal dimensions of 12 feet by 12 feet or 144 square feet in area. Should the stall be larger or smaller, these quantities should be adjusted in proportion.

While the above description contains many specificities, the reader should not construe these as limitations on the scope of the invention, but merely as exemplifications of preferred embodiments thereof. Those skilled in the art will envision many other possible variations within its scope. Accordingly, the reader is requested to determine the scope of the invention by the appended claims and their legal equivalents, and not by the examples which have been given.

I claim:

1. A layered bedding configuration for a stall used as a shelter for an animal, capable of reducing odors and comprising:
a) a first film of enzyme solution on the floor and on the walls;
b) a first layer of enzymatic-bacterial composition called enzyme powder on top of said first film of enzyme solution, said enzyme powder comprises hydrolyzing enzymes, bacteria and bacterial growth medium;
c) a layer comprising rice hulls on top of said first layer of enzyme powder, said layer of rice hulls being gradually crushed as said animal moves in said stall;
d) a second layer of enzyme powder;
e) a second film of enzyme solution on top of said second layer of enzyme powder;
wherein said enzyme solution and said enzyme powder act on fecal and urinary materials produced by said animal to reduce odor, and said layer of rice hulls increases its absorbency as a function of the degree that it is crushed by said animal, said crushing degree being in turn a function of the time said animal occupies said stall.

2. A bedding configuration as in claim 1 wherein said hydrolyzing enzymes comprise at least one member of the group consisting of alpha amylase, beta amylase, pentosanase, gluco-amylase, cellulase, lactase, pancrease, protease, phosphorilase, pullulanase, hemicellulase, pectynase, beta-glucanase, and carbohydrase;

3. A bedding configuration as in claim 1 wherein said enzyme solution comprise at least one member of the group consisting of alpha amylase, beta amylase, pentosanase, gluco-amylase, cellulase, lactase, pancrease, protease, phosphorilase, pullulanase, hemicellulase, pectynase, beta-glucanase, carbohydrase and surfactants;

4. A bedding configuration as in claim 1 wherein said bacteria comprise Micrococcus.

5. A bedding configuration as in claim 1 wherein said bacteria comprise Bacillus.

6. A bedding configuration as in claim 1 wherein said bacterial growth medium comprises at least one member of the group consisting of maltodextrin, AGAR broth medium, Lithothamnium Calcareum, DNA and RNA nucleic acids, extracts of Laminariae Stipites, and extracts of Fucus Vesiculosus.

7. A bedding configuration as in claim 1 wherein said first film of enzyme solution has diffused up into said first layer of enzyme powder and said layer of rice hulls, and wherein said first layer of said enzyme powder is intermingled with said rice hulls;

8. A bedding configuration as in claim 1 wherein said second film of enzyme solution has diffused down into said second layer of enzyme powder and said layer of rice hulls, and wherein said second layer of said enzyme powder is intermingled with said rice hulls;

9. A bedding configuration as in claim 1 wherein said enzyme powder includes at least the precursor of one class of enzymes, one bacterial species, and mineral salts, characterized in that the said pro-enzymes, precursors of the active enzymatic form, comprise amylases, lipases, cellulases, and proteases, and at least one bacterial species selected from the group consisting of cocci and bacilli;

10. A bedding configuration as in claim 1 wherein said first film and said second film of enzyme solution each are produced by the application of an amount of said enzyme solution ranging from 4 to 20 volume ounces for staid stall, said stall being nominally a standard 144 square feet, said amount varying in proportion to said stall actual size.

11. A bedding configuration as in claim 1 wherein said first film and said second film of enzyme solution each are produced by the application of an amount of said enzyme solution ranging from 8 to 10 volume ounces for staid stall, said stall being nominally a standard 144 square feet, said amount varying in proportion to said stall actual size.

12. A bedding configuration as in claim 1 wherein said first layer of enzyme powder and said second layer of enzyme powder each are produced by the application of an amount of said enzyme powder ranging from 2 to 20 weight ounces for staid stall, said stall being nominally a standard 144 square feet, said amount varying in proportion to said stall actual size.

13. A bedding configuration as in claim 1 wherein said first layer of enzyme powder and said second layer of enzyme powder each are produced by the application of an amount of said enzyme powder ranging from 7 to 10 weight ounces for staid stall, said stall being nominally a standard 144 square feet, said amount varying in proportion to said stall actual size.

14. A bedding configuration as in claim 1 wherein a board is placed on its edge on the floor across the entrance of said stall, whereby said board retains said bedding configuration within said stall.

15. A method of setting up the layered bedding configuration of claim 1 in a stall, comprising:
a) removing old bedding material if said old material occupies said stall;
b) spraying the floor and walls of said stall with said liquid enzyme solution;
c) spreading a first layer of said enzyme powder on the floor;
d) spreading on top of said first layer of enzyme powder a layer comprising said rice hulls;
e) placing a board on its edge across the entrance of said stall to prevent said rice hulls from spilling outside of said stall;
f) leveling said rice hulls layer;
g) spreading a second layer of enzyme powder on top of said layer of rice hulls; and
h) spraying said liquid enzyme solution on top of said layer of rice hulls.

16. A method as of claim 15 wherein the amount of said layer of enzyme powder ranges from 2 weight ounces to 10 ounces for staid stall, said stall being nominally a standard 144 square feet, said amount varying in proportion to said stall actual size.

17. A method as of claim 14 wherein the amount of material of said layer of enzyme powder ranges from 8 to 10 weight ounces for staid stall, said stall being nominally a standard 144 square feet, said amount varying in proportion to said stall actual size.

18. A method as of claim 14 wherein said rice hulls layer is approximately 5 to 6 inches thick.

19. A method as of claim 14 wherein said board is approximately 5 to 6 inches wide.

20. A method of maintaining the layered bedding configuration of claim 1 comprising:
a) removing fecal material produced by said animal;
b) adding said rice hulls to compensate for the compaction of said bedding configuration caused by said animal;
c) spreading enzyme powder on top of said bedding configuration to restore said second layer of enzyme powder;
d) spraying said enzyme solution to restore second film of enzyme solution;
e) removing wet spots generated in said bedding configuration when said animal urinates.

21. A method as in claim 19 wherein said removing of said fecal matter is done twice a day.

22. A method as in claim 19 wherein said adding said rice hulls and spreading said enzyme solution is done weekly.

23. A method as in claim 19 wherein said spraying of enzyme solution is done monthly.

24. A method as in claim 19 wherein said removing wet spots is done as needed, said wet spot consisting of material soaked by urine from said animal, step of removing said wet spot material comprising:
a) determining that said wet spot needs to be removed when it has grown to such a size that said wet material is barely visible below the top surface of said bedding configuration;
b) exposing said wet spot by brushing away from it surrounding said rice hulls from said bedding configuration;
c) removing only said wet material, making a hole in said bedding configuration and exposing the floor of said stall;
d) spreading a first layer of said enzyme powder on said exposed floor to reconstitute said first layer of enzyme powder;
e) leveling the surface of said bedding configuration to cover up said hole;
f) spreading additional said rice hulls over the surface of said bedding configuration to compensate for the removal of said wet material and for the loss of volume caused by the compaction of said rice hulls by said animal;
g) spreading a second layer of said enzyme powder on top of said bedding configuration to restore said second layer of enzyme powder;
h) spraying said enzyme solution to restore said second film of enzyme solution.

25. A method as in claim 24 wherein said spreading a first layer of said enzyme powder and spreading a second layer of said enzyme powder, each requires an amount of said enzyme powder between 2 and 20 weight ounces for staid stall, said stall being nominally a standard 144 square feet, said amount varying in proportion to said stall actual size.

26. A method as in claim 24 wherein said spraying said enzyme solution requires an amount of said enzyme solution between 2 and 20 volume ounces for staid stall, said stall being nominally a standard 144 square feet, said amount varying in proportion to said stall actual size.
